(19) United States
(12) Patent Application Publication
(10) Pub. No.: US 2014/0135929 A1
(43) Pub. Date: May 15, 2014

(54) IMPLANT ASSEMBLY

(71) Applicant: Terumo Kabushiki Kaisha, Tokyo (JP)

(72) Inventors: Yasuyuki HONMA, Kanagawa (JP); Yuji NAKAGAWA, Kanagawa (JP); Suguru HATA, Kanagawa (JP)

(73) Assignee: Terumo Kabushiki Kaisha, Tokyo (JP)

(21) Appl. No.: 14/144,967

(22) Filed: Dec. 31, 2013

Related U.S. Application Data
(63) Continuation of application No. PCT/JP2012/064925, filed on Jun. 11, 2012.

(30) Foreign Application Priority Data
Jul. 4, 2011 (JP) 2011-148576

(51) Int. Cl. A61F 2/46

(52) U.S. Cl. CPC A61F 2/4611
USPC 623/17.12; 623/17.16

(57) ABSTRACT

An implant assembly includes an implant configured to be placed in a living body; an elongated body that includes a pulling unit, the pulling unit being configured such that a portion of the pulling unit is locatable outside the living body when the implant is placed in the living body, and the pulling unit being connectable to the implant and pullable to move the implant to a placement position; and a guide device configured to allow for an a percutaneous introduction of the elongated body into the living body.

### CROSS-REFERENCE TO RELATED APPLICATIONS

This application is a continuation application filed under 35 U.S.C. 111(a) claiming the benefit under 35 U.S.C. §§120 and 365(c) of PCT International Application No. PCT/JP2012/064925 filed on Jun. 11, 2012, which is based upon and claims the benefit of priority of Japanese Application No. 2011-148576 filed on Jul. 4, 2011, the entire contents of which are hereby incorporated by reference in their entireties.

### BACKGROUND

The present disclosure relates to an implant assembly that includes an implant which is positioned and placed in a living body.

In the medical field, techniques relating to implants that are placed in living bodies and various techniques relating to methods for placing the implants in the living bodies are known. For example, a medical instrument that includes an implant that is placed between spinous processes of the living body to treat lumbar spinal canal stenosis and a head section to which a tool and an engagement member or a connection member can be attached to smoothly perform an introduction of the implant into between the spinous processes is known (refer to PCT Publication No. WO07/018114).

### SUMMARY

Various implants are provided to be used at positions of living bodies. Because placement positions of the implants vary depending on the sites of the living bodies to which the implants are applied, it is necessary to perform appropriate positioning at each of the sites to which the implants are applied. Particularly, in order to perform the positioning via a minimally invasive method so as to reduce a load on the living body, it is desirable to minimize a wound hole for the introduction into the living body and perform an operation in a swift and safe manner.

An object of embodiments of the present invention is to provide an implant assembly with which the implant can be positioned in the placement position through a simple operation outside the living body regardless of the site to which the implant is applied.

In one embodiment of the present invention, an implant assembly includes an implant that is placed in a living body, an elongated body that includes a pulling unit which is positioned outside the living body and is pulled to move the implant to a placement position, and is connected to the implant, and guide means for assisting in a percutaneous introduction of the elongated body into the living body.

In one aspect, the elongated body includes at least a first elongated piece and a second elongated piece that are connected to the implant in a pair, and a pulling unit of the first elongated piece and a pulling unit of the second elongated piece are respectively introduced out of the living body from different positions of the living body.

In one aspect, the guide means includes a puncture member that is percutaneously punctured into the living body, the implant is connected to the puncture member via the elongated body and is introduced into the living body as the puncture member is punctured, and the elongated body is introduced into the living body with the implant in a state of being connected to the implant.

In one aspect, the puncture member has a body section into which the implant can be inserted, and the implant can be introduced into the living body in a state of being inserted into and held in the body section.

In one aspect, the guide means includes a tubular guide member which has a lumen into which the elongated body can be inserted and opening sections that are respectively formed on one end side and the other end side of the lumen.

In one aspect, the implant includes an attachment section to which the elongated body is attached in a connectible and separable manner.

In one aspect, the attachment section has a hook by which the elongated body is hooked and held, and the implant and the elongated body can be connected to each other as a part of the elongated body is hooked on the hook.

In one aspect, the elongated body is annularly hooked by and attached to the hook and the implant and the elongated body can be separated from each other by cutting the part of the elongated body.

In one aspect, the implant is an expansion member that can be expansively deformed by an introduction of the filling material and is introduced into the living body in a state before the expansive deformation.

According to the implant assembly of embodiments of the present invention, the implant can be positioned at the placement position through a simple operation in which the pulling unit of the elongated body connected to the implant placed in the living body is pulled outside the living body. In this manner, a placement operation for the implant can be performed in a swift and minimally invasive manner.

### BRIEF DESCRIPTION OF DRAWINGS

FIGS. 1A and 1B are views showing an overall configuration of an implant assembly according to a first embodiment of the present invention. FIG. 1A is a view showing a state where an implant is inserted into a body section of a puncture member of the implant assembly, and FIG. 1B is an enlarged view of the implant shown in FIG. 1A.

FIGS. 2A and 2B are views showing the overall configuration of the implant assembly. FIG. 2A is a view showing a state where the implant is taken out of the body section of the puncture member of the implant assembly, and FIG. 2B is an enlarged view of the implant shown in FIG. 2A.

FIG. 3 is a view showing a site in a living body to which the implant assembly may be applied.

FIGS. 4A and 4B are schematic views illustrating a method of using the implant assembly. FIG. 4A is a view showing a state before the implant is introduced into the living body, and FIG. 4B is a view showing a state where the implant is introduced into the living body with the puncture member.

FIGS. 5A and 5B are schematic views illustrating the method of using the implant assembly. FIG. 5A is a view showing an operation to move the implant to a placement position in the living body, and FIG. 5B is an arrow view as viewed from the arrow 5B direction of FIG. 5A.

FIGS. 6A and 6B are schematic views illustrating the method of using the implant assembly. FIG. 6A is a view showing an operation to separate an elongated body from the implant and an operation to expansively deform the implant at the placement position, and FIG. 6B is an arrow view as viewed from the arrow 6B direction of FIG. 6A.

FIGS. 7A and 7B are schematic views illustrating the method of using the implant assembly. FIG. 7A is a view showing a state where the expansively deformed implant is placed at the placement position, and FIG. 7B is an arrow view as viewed from the arrow 7B direction of FIG. 7A.

FIG. 8 is an overall configuration view illustrating a modified example of the puncture member.

FIGS. 9A-9C are views illustrating a modified example of an attachment section of the implant. FIG. 9A is a view showing an example of the shape of a hook, FIG. 9B is a view showing the example of the shape of the hook and an example of the shape of an end section of the elongated body, and FIG. 9C is a partially enlarged view of FIG. 9B.

FIGS. 10A-10C are views illustrating an implant assembly according to a second embodiment of the present invention. FIG. 10A is a schematic view showing a state where a tubular guide member of the implant assembly is in use, and each of FIGS. 10B and 10C is a partially enlarged view illustrating the form of connection between an implant and an elongated body.

### DETAILED DESCRIPTION

### First Embodiment

Hereinafter, embodiments of the present invention will be described based on the accompanying drawings. In the description of the drawings, the same reference numerals are used to designate the same components and redundant descriptions thereof are omitted. In some cases, dimensional ratios in the drawings are exaggerated and are different from the actual ratios for convenience of description.

### Overall Configuration of Implant Assembly

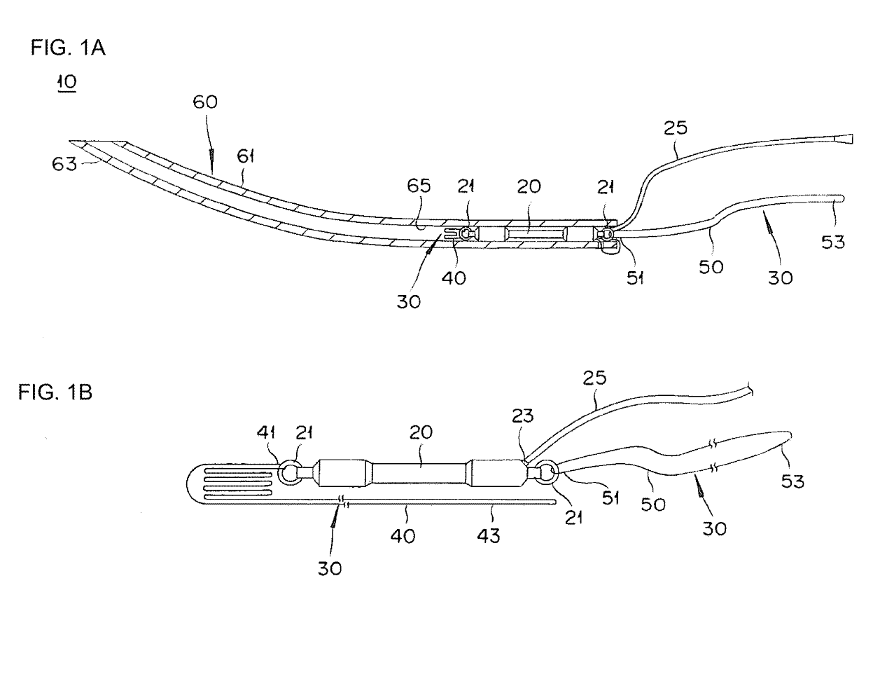

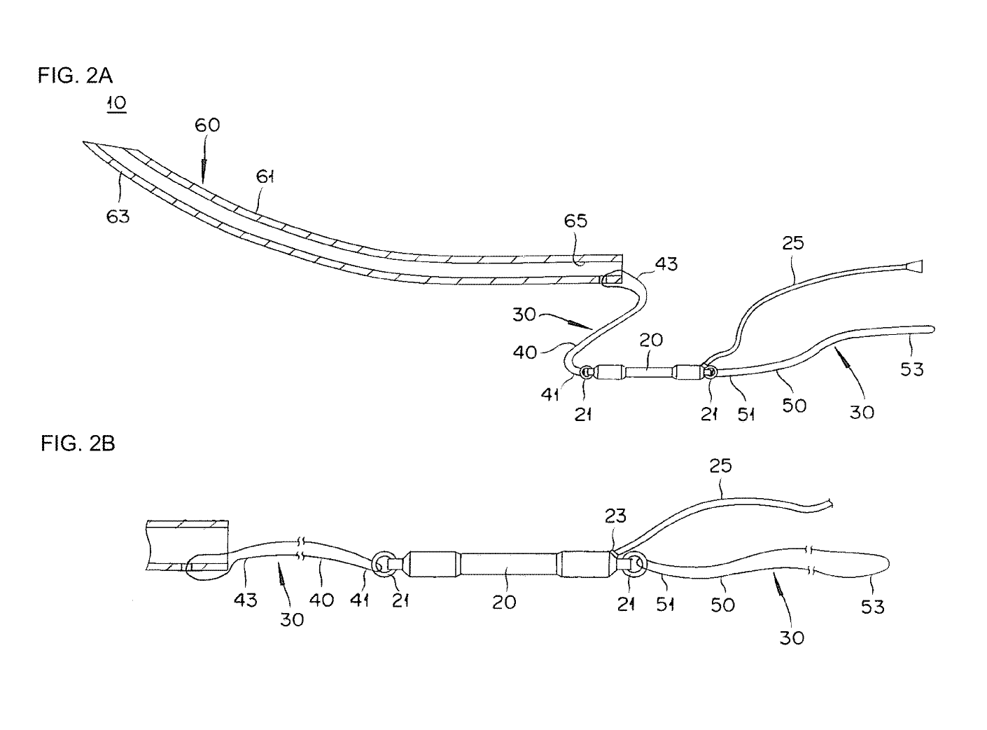

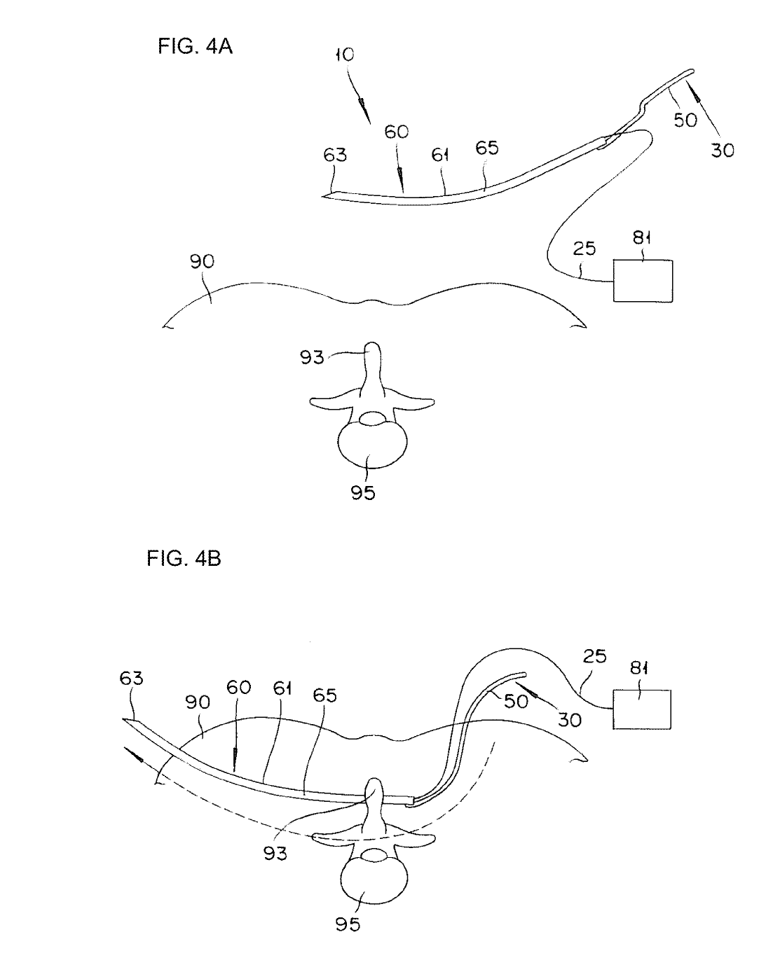

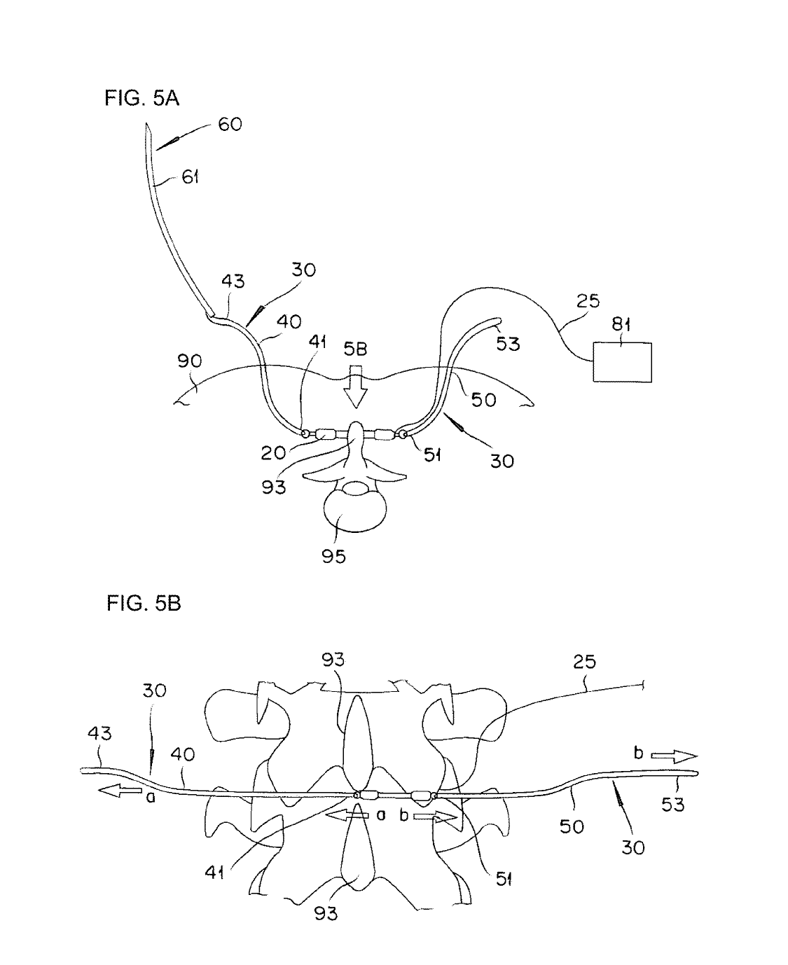

Referring to FIGS. 1 and 2, an implant assembly 10 according to an embodiment includes an implant 20 that is placed in a living body 90, an elongated body 30 that has pulling units 43 and 53 which are positioned outside the living body 90 and are pulled to move the implant 20 to a placement position, and is connected to the implant 20, and guide means 60 for assisting in a percutaneous introduction of the elongated body 30 into the living body 90 (refer also to FIGS. 4 and 5).

The implant 20 is not particularly limited to an expansively deformable expansion member but may be one of various other implantable devices. For example, the implant 20 may be a device that is placed in a living body for treatment and drug administration purposes, such as ports, IC tags (IC chips), drug administration pumps, pacemakers, nerve stimulation devices, defibrillators, bone spacers, and spinal instrumentations. In one embodiment, the implant 20 is a spacer that is used for spacing between spinous processes, which is used for the purpose of treating lumbar spinal canal stenosis (LSCS).

A lumbar spine, which is an organ of the living body, has a vertebral body as a front half and a vertebral arch as a rear half which are interconnected through a pedicle of vertebral arch, with the vertebral arch formed with processes such as a spinous process, a mamillary process and an accessory process, and normally has a slightly forwardly curved shape. In addition, since adjacent lumbar vertebrae are interconnected through an intervertebral disk, a certain lumbar vertebra and the adjacent vertebra, for example, are prevented from getting out of alignment with each other by an intervertebral disk and a facet joint. When a load is repeatedly exerted on the lumbar vertebrae due to sports or the like to cause a fatigue fracture, there would result lumbar spondylolysis, in which the lumbar vertebrae are separated at the vertebral arch part, or lumbar degenerated spondylolisthesis, in which it becomes difficult to fix the upper lumbar vertebra due to degeneration of the intervertebral disk or the shape of the facet joint and a slippage of the lumbar vertebrae is thereby caused. Further, a severe slip of lumbar vertebrae may cause stenosis of the vertebral canal, possibly leading to intermittent claudication, which is a symptom of lumbar spinal canal stenosis. According to the embodiment, the implant 20 that functions as the spacer between the spinous processes is positioned and placed, and lumbar spinal canal stenosis can be treated in a minimally invasive manner without performing an operation with a large load on the living body, such as an incision surgery in which the living body is incised, or screwing in the living body.

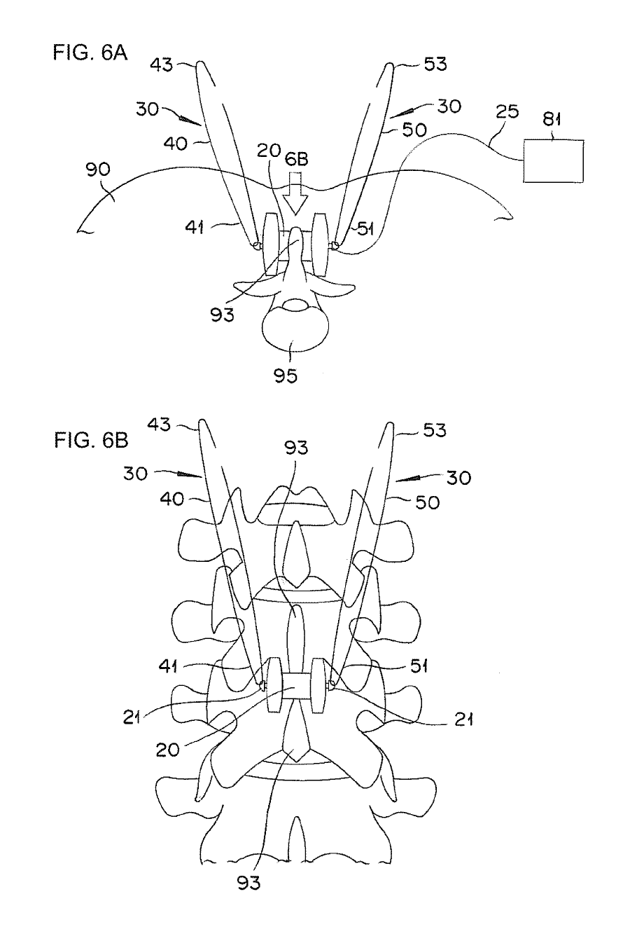

The implant 20 is formed from an expansion member that can be expansively deformed as a tilling material (which may be a fluid or a solid) is introduced. The implant 20 is introduced into the living body 90 in a state before the expansive deformation (refer to FIG. 5), and then is expansively deformed after the positioning to the placement position (refer to FIG. 6). The implant 20 is expansively deformed to have a substantially H-shaped appearance (dumbbell shape), and is placed in the living body 90 in that shape. A movement of the implant 20 after the placement can be prevented by allowing spacing between spinous processes 93 in a central part of the implant 20 and, in addition, by allowing the spinous processes 93 to be pinched in enlargement parts that are positioned on both sides of the central part.

The material of the implant 20 that is used as the spacer between the spinous processes is not particularly limited insofar as the material is durable to external pressures resulting from movements of a tissue such as the spinous processes and the vertebral body. Preferable examples of the material include polyvinyl chloride, a thermoplastic elastomer such as a polyurethane elastomer, a styrene-ethylene-butylene-styrene copolymer (SEBS), and a styrene-ethylene-propylene-styrene copolymer (SEPS), a thermoplastic resin such as nylon and PET, or a thermosetting resin such as rubber and a silicone elastomer, and particularly, a porous material such as non-woven fabric, woven fabric, knitted fabric, and ePTFE. Also, these can be used in an appropriate combination.

Also, the implant 20 of the present invention is not particularly limited to the expansively deformable expansion member that is described above. In this case, the material of the implant 20 that is used as the spacer between the spinous processes may be any material that is durable to the external pressures resulting from movements of the tissue such as the spinous processes and the vertebral body, preferable examples of which include a metallic material such as SUS and titanium, particularly a ceramic material such as hydroxyapatite, a bone cement, and calcium phosphate.

A tube 25 is connected to the implant 20, and the implant 20 is expansively deformed by the introduction of the filling material that is formed of the fluid or the solid via the tube 25. Preferable examples of the fluid include any material that is a fluid at the time of injection and hardens after the injection, or material that is a fluid at the time of injection and does not harden after the injection.

Preferably, the material that is a fluid at the time of injection and hardens after the injection and the material that is a fluid at the time of injection and does not harden after the injection have at least one of the following characteristics: (a) to be safe to a patient; (b) to cause little or no damage to tissues; (c) to harden at a temperature (approximately 35° C. to 42° C.) close to the body temperature of the patient; (d) to be free of contraction or expansion and be capable of maintaining the shape upon hardening; (e) to harden within one to 60 minutes, preferably five to 30 minutes, and more preferably 10 minutes, after the injection; (f) to allow use of water, a buffer solution, physiological saline, a contrast agent, or oils and fats such as olive oil and castor oil, as a solvent therefor.

Specific examples of the material (hardening material) that is a fluid at the time of injection and hardens after the injection include (g) a two-part type crosslinking polymer, (h) a hot melt adhesive, (i) a urethane elastomer, (j) a photo-curing resin, (k) an acrylic resin, (l) a bone cement, (m) a solution which is crystallized in response to an external stimulus.

Preferably, the two-part type crosslinking polymer of (g) above is a combination of an aromatic diepoxide resin or an aliphatic diepoxide resin with an amine compound.

Examples of the hot melt adhesive of (h) above include a combination of a material capable of being hardened by reaction with water with water, or adhesives based on ethylene-vinyl acetate copolymer (EVA), polyolefin (PO), polyamide (PA), synthetic rubber (SR), acryl (ACR), and polyurethane (PUR; moisture-hardening type).

Preferably, the urethane elastomer of (i) above is a polymer which is derived from a polyol and an aromatic polyisocyanate.

Examples of the photo-polymerizable monomer of (j) above include acrylate, methacrylate, and ethylenically unsaturated carboxylic acid. A polymerization accelerator, a crosslinking agent, a photo-polymerization initiator or the like can be used as required.

Examples of the acrylic resin of (k) above include those obtained by polymerization, according to known methods, of such a monomer as methyl(meth)acrylate, ethyl(meth)acrylate, butyl(meth)acrylate, n-hexyl(meth)acrylate, cyclohexyl(meth)acrylate, 2-ethylhexyal(meth)acrylate, n-octyl(meth)acrylate, nonyl(meth)acrylate, decyl(meth)acrylate, (meth)acrylic acid, glycidyl(meth)acrylate, vinyl acetate, styrene, α-methylstyrene, (meth)acrylamide, and (meth)acrylonitrile.

The bone cement of (l) above is prepared, for example, by mixing a powder of polymethyl methacrylate, a methylmethacrylate-styrene copolymer, benzoyl peroxide, barium sulfate or the like with a solvent such as methyl methacrylate, N,N-dimethyl-p-toluidine, hydroquinone, and the like. Alternatively, a dental cement that is hardened through an acid-base reaction between zinc oxide and phosphoric acid, an organic-inorganic composite in which a solvent is mixed with sodium alginate, sodium phosphate, calcium chloride and the like to prepare sodium alginate that is an organic material and calcium phosphate that is an inorganic material are prepared, and the like can also be used.

Examples of the solution which is crystallized in response to an external stimulus of (m) above include an aqueous solution prepared by dissolving sodium acetate, sodium chloride or the like. Examples of the external stimulus include a physical shock, heat, light, electricity, and an ultrasonic wave.

After the fluid is injected into the implant 20, the fluid hardens with time, and thus the implant 20 can function as the spacer between the spinous processes 93 over a long period of time in the expansively deformed state.

Specific examples of the material that is a fluid at the time of injection and does not harden after the injection include a liquid (physiological saline, a contrast agent, a medicinal liquid, and the like); a dilatant fluid which is a material varying in hardness according to pressurization speed; a gel (gelatin, agar-agar, starch, and the like); a silicone sealant; a biomaterial or a bio-absorbable material; and slimes (mixture of polyvinyl alcohol and boric acid). After the injection into the implant 20, the state in which the implant 20 is placed is maintained without being damaged by a body motion, and thus the implant 20 can function as a spacer between the spinous processes 93 over a long period of time in the expansively deformed state.

Also, specific examples of the material already hardened at the time of injection include a solid (a super elastic wire, a coil, and the like) such as metal. After the injection into the implant 20, the state in which the implant 20 is placed is maintained without being damaged by the body motion, and thus the implant 20 can function as the spacer between the spinous processes 93 over a long period of time in the expansively deformed state.

The implant 20 has a connection port 23 to which one end of the tube 25 is connected. The tube 25 is liquid-tightly connected to the connection port 23 by screwing, fitting, or the like. The form of the connection is not particularly limited, but can be appropriately selected such that the filling material can be supplied to the implant 20 and the tube 25 can be separated from the implant 20.

The implant 20 has attachment sections 21 to which the elongated body 30 is attached in a connectible and separable manner. The attachment sections 21 are constituted by hooks by which the elongated body 30 is hooked and held. The hooks have a ring shape that has a hole into which the elongated body 30 is inserted, and are disposed on both side sections of the implant 20. Grooving (not shown in the drawing) is performed on the attachment sections 21 so that an introduction position can be easily detected by ultrasonic waves after the introduction into the living body.

The elongated body 30 has a first elongated piece 40 and a second elongated piece 50 that are connected to the implant 20 in a pair.

The first elongated piece 40 is constituted by a string-shaped member that has a connection section 41 which is connected to the implant 20, and a pulling unit 43 which is positioned outside the living body 90. The connection section 41 is constituted by a part of the first elongated piece 40 that is hooked on the attachment section 21 of the implant 20, and the pulling unit 43 is constituted by a part of the elongated piece 40 that extends to the connection section 41 and is introduced out of the living body 90.

The second elongated piece 50 that is used in a pair with the first elongated piece 40 has a similar configuration to the first elongated piece 40, that is, is constituted by a string-shaped member in entirety, and has a connection section 51 that is connected to the implant 20, and the pulling unit 53 that is positioned outside the living body.

The material of the elongated body 30 is not particularly limited, but it is preferable that a material that has biocompatibility be used. Preferable examples include polyvinyl chloride, a thermoplastic elastomer such as a polyurethane elastomer, a styrene-ethylene-butylene-styrene copolymer (SEBS), and a styrene-ethylene-propylene-styrene copolymer (SEPS), a thermoplastic resin such as nylon and PET, or a thermosetting resin such as rubber and a silicone elastomer, and particularly, a fibrous material such as a silk thread, a cotton thread, and cellulose fiber, and a metallic material such as an SUS wire, a copper wire, a titanium wire, and a Nitinol wire. Also, these can be used in an appropriate combination.

Also, the appearance and dimensions of the elongated body 30 are not particularly limited but, for example, it is preferable that the length be sufficient to be introduced out of the living body 90 from the placement position. Also, it is preferable that the diameter be small in view of facilitating the movement in the living body 90 and reducing the load on the living body 90 resulting from the movement.

The pulling unit 43 of the first elongated piece 40 and the pulling unit 53 of the second elongated piece 50 arc respectively introduced out of the living body 90 from different positions of the living body 90 (refer to FIG. 5) so that the implant 20 can be moved in a pulling direction of the pulling unit 43 of the first elongated piece 40 and, in addition, the implant 20 can be moved in a pulling direction of the pulling unit 53 of the second elongated piece 50 after the implant 20 is introduced into the living body 90.

The first elongated piece 40 is hooked in an annular shape on and attached to the attachment section 21 of the implant 20. Likewise, the second elongated piece 50 is hooked in an annular shape on and attached to the other attachment section 21 of the implant 20.

The guide means 60 has a puncture member 61 that is percutaneously punctured into the living body 90. The puncture member 61 is constituted by a puncture needle that has a needle section 63 whose tip section is formed into a sharp end and a body section 65 into which the implant 20 can be inserted.

The needle section 63 of the puncture member 61 has a generally curved shape. By adopting the appearance, the puncture member 61 can be moved along a curved path that goes through an insertion position into the living body 90, between the spinous processes 93, and the introduction position outside the living body 90 (the path is shown with the dashed line of FIG. 4B).

The implant 20 is inserted into the body section 65 of the puncture member 61 in a state before the expansive deformation. A part of the first elongated piece 40 is partially connected to the puncture member 61 and, in addition, is inserted into the body section 65 in a folded state. As shown in FIG. 2, the pulling unit 43 of the first elongated piece 40 is connected to the vicinity of an open end section of the puncture member 61. The form of the connection is not particularly limited. The annular first elongated piece 40 may pass through a small hole bored in the vicinity of an open end of the puncture member 61 as shown in the drawing, or a simple method such as adhesion and welding may be used in an inner section of the puncture member 61 without using such a small hole.

The implant 20 is connected to the puncture member 61 via the first elongated piece 40. When the implant 20 is introduced, the needle section 63 that is introduced ahead of the implant 20 passes between the adjacent spinous processes 93, and thus the implant 20 that is held in the body section 65 and the first elongated piece 40 that is connected to the puncture member 61 are respectively guided between the spinous processes 93.

### Method for Placing Implant

Next, a method for placing the implant 20 by using the implant assembly 10 will be described.

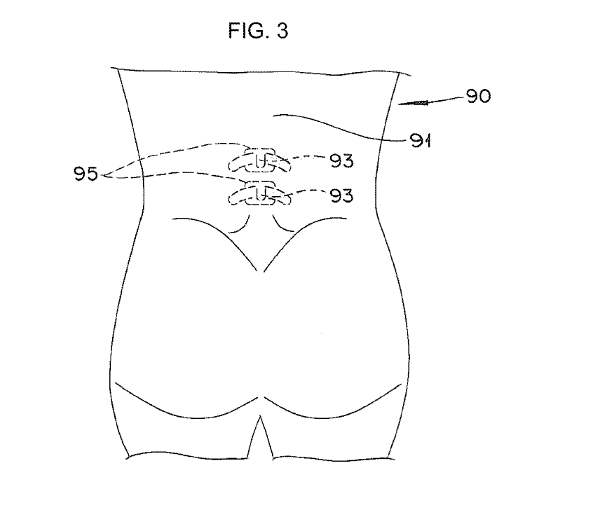

FIG. 3 is a simplified view of the living body 90 where the implant 20 is placed. The implant 20 is percutaneously introduced into the living body 90 from the back 91 of the living body 90, and is placed after being positioned at the placement position between the spinous processes 93. As shown in the drawing, the spinous processes 93 are arranged apart from each other in an extension direction of a vertebral body 95.

The method for placing the implant according to the embodiment includes (i) a step of introducing the implant into the living body, (ii) a step of introducing the elongated body that is connected to the implant into the living body, (iii) a step of positioning the pulling unit of the elongated body outside the living body, and (iv) a step of moving the implant to the placement position by pulling the pulling unit out of the living body.

First, referring to FIG. 4A, the implant assembly 10 is prepared in a state where the implant 20 is inserted into the body section 65 of the puncture member 61. The tube 25 is connected to the implant 20 in advance.

Next, referring to FIG. 4B, the puncture member 61 is introduced into the living body 90. A puncture site of the living body 90 may be marked or the like before the puncturing.

The implant 20 is introduced into the living body 90 in a state of being inserted into and held in the body section 65 of the puncture member 61. The first elongated piece 40 and the second elongated piece 50 that are connected to the puncture member 61 are introduced into the living body 90 with the puncture member 61. In this case, a tip side of the needle section 63 of the puncture member 61 penetrates the living body 90. The pulling unit 53 of the second elongated piece 50 is not introduced into the living body 90 but is positioned outside the living body 90.

Since the implant 20 is connected to the puncture member 61 via the elongated body 30, the implant 20 and the first elongated piece 40 can be guided along the path in the living body 90 through which the puncture member 61 passes. Also, since the implant 20 is inserted into the body section 65 of the puncture member 61, the implant 20 can be smoothly introduced into the living body 90 regardless of the appearance of the implant 20.

In this manner, the step (i) includes a step of connecting the implant in advance, via the elongated body, to the puncture member that is punctured into the living body and introducing the implant and the elongated body into the living body with the puncturing of the puncture member. Also, the step (i) includes a step of introducing the implant into the living body in a state where the implant is inserted into and held in the body section of the puncture member. In addition, the step (i) includes a step of introducing the implant that can be expansively deformed by the introduction of the filling material into the living body in a state before the expansive deformation. Also, the step (i) includes a step of introducing the pulling unit of the first elongated piece that is connected to the implant outside the living body out of the living body from an introduction direction front side of the implant and introducing the pulling unit of the second elongated piece that is connected to the implant in a pair with the first elongated piece out of the living body from an introduction direction rear side of the implant.

Referring to FIG. 5A, the tip side of the needle section 63 of the puncture member 61 is pulled outside the living body 90 while the pulling unit 53 of the second elongated piece 50 is gripped outside the living body 90. The pulling unit 43 of the first elongated piece 40 is introduced out of the living body 90 while the implant 20 is drawn out of the body section 65 of the puncture member 61. The pulling unit 53 of the second elongated piece 50 is positioned outside the living body 90 without being introduced into the living body 90.

In this manner, the step (ii) includes a step of introducing the first elongated piece into the living body, introducing the pulling unit of the first elongated piece out of the living body, introducing the second elongated piece into the living body, and leaving the pulling unit of the second elongated piece outside the living body by inserting the puncture member into the living body in a state where the first elongated piece and the second elongated piece are respectively connected to the puncture member.

Referring to FIG. 5B, the implant 20 that is introduced into the living body 90 is moved to the placement position by respectively pulling the pulling unit 43 of the first elongated piece 40 and the pulling unit 53 of the second elongated piece 50 outside the living body 90. The implant 20 can be slid in two directions (arrow a and b directions in the drawing), along the path between the spinous processes 93 through which the puncture member 61 passes, and can be positioned. The positioning operation can be performed by pulling the tube 25 that is connected to the implant 20 instead of the second elongated piece 50 or with the second elongated piece 50. In this case, detailed positioning to the placement position can be performed based on, for example, an image detected by ultrasonography.

Compared to a case where the positioning operation is performed after the expansion, the implant 20 can be smoothly moved to the placement position since the positioning operation is performed before the expansion of the implant 20.

In this manner, the step (iv) includes a step of positioning the implant that is introduced into the living body to a predetermined placement position by pulling the pulling unit of the first elongated piece outside the living body and pulling the pulling unit of the second elongated piece outside the living body. Also, the step includes (iv) a step of performing the positioning of the implant by pulling the tube that is connected to the implant outside the living body instead of the second elongated piece or with the second elongated piece. Also, the step (iv) includes a step of moving the implant to the placement position in a state before the expansive deformation.

Referring to FIGS. 6A and 6B, the filling material is supplied to the implant 20 via the tube 25 after the positioning is performed. The supply of the filling material to the implant 20 via the tube 25 is performed by using a filling material supply device 81 that is an external device. For example, in a case where a fluid is used as the filling material, a known fluid pump or the like that has the function of pumping the fluid as the filling material can be used as the filling material supply device 81.

In this manner, after the step (iv), a step (v) of expansively deforming the implant 20 that is positioned at the placement position is performed.

Next, an annular part of the first elongated piece 40 is cut and the first elongated piece 40 is taken out of the living body 90. The cutting can be performed by using a cutting instrument such as a pair of scissors. Likewise, an annular part of the second elongated piece 50 is cut and the second elongated piece 50 is taken out of the living body 90. The implant 20 and the elongated body 30 can be separated from each other through a simple operation in which the part of the first elongated piece 40 and the part of the second elongated piece 50 are respectively cut.

In this manner, after the step (v), a step (vi) of separating the elongated body from the implant is performed. Also, the step (vi) includes a step of separating the implant and the elongated body from each other by cutting the part of the elongated body that is hooked in an annular shape on the hook of the implant. In a case where the expansion body is applied to the implant, the step (vi) is performed after the step (v). However, in a case where an implant other than the expansion body is placed, the step of separating the elongated body is performed without performing the step (v).

The tube 25 that is connected to the implant 20 is appropriately separated from the implant 20 after the implant 20 is expansively deformed.

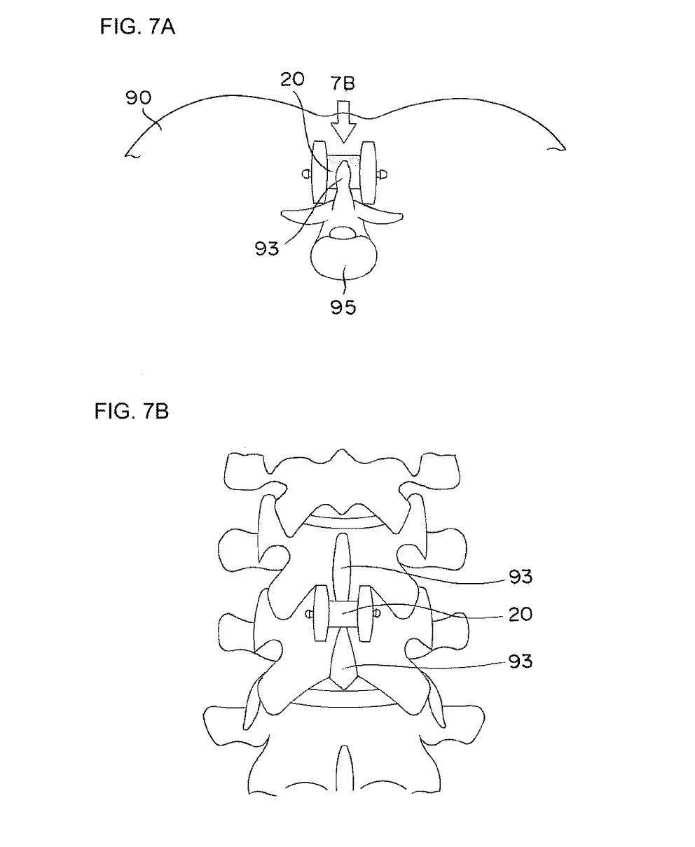

Referring to FIGS. 7A and 7B, the implant 20 in the expansive deformation state is placed between the spinous processes 93. The implant 20 functions as the spacer allowing spacing between the adjacent spinous processes 93. In this manner, lumbar spinal canal stenosis can be treated without performing an operation with a large load on the living body 90 such as an incision surgery or screwing in the living body 90 for the implant.

As described above, according to the implant assembly 10 of the embodiment, the implant 20 can be positioned at the placement position through a simple operation in which the pulling units 43 and 53 of the elongated body 30 connected to the implant 20 placed in the living body 90 are pulled outside the living body 90. In this manner, a placement operation for the implant 20 can be performed in a swift and minimally invasive manner.

Also, the implant 20 that is introduced into the living body 90 can be slid in the two directions and positioned by respectively pulling the pulling unit 43 of the first elongated piece 40 and the pulling unit 53 of the second elongated piece 50 outside the living body 90. Accordingly, the positioning operation can be performed more simply and accurately.

Also, the puncture member 61 and the implant 20 are connected to each other via the first elongated piece 40, and the implant 20 and the first elongated piece 40 are introduced into the living body 90 with the puncture member 61. Accordingly, the implant 20 and the first elongated piece 40 can be guided along the path in the living body 90 through which the puncture member 61 passes. In addition, an operation to introduce the implant 20 and an operation to introduce the first elongated piece 40 can be performed in a single process. Accordingly, the placement operation for the implant 20 can be performed in a swifter manner

Also, the implant 20 is introduced into the living body in a state of being inserted into and held in the body section 65 of the puncture member 61. Accordingly, the implant 20 can be smoothly introduced into the living body 90 regardless of the appearance of the implant 20, and the introduction can be performed in a minimally invasive manner.

Also, the attachment sections 21, to which the elongated body 30 is attached in a connectible and separable manner, is disposed in the implant 20. Accordingly, in the medical field, a worker can easily and manually connect and separate the implant 20 and the elongated body 30 with and from each other.

Also, the attachment section 21 is constituted by the hook so that the elongated body 30 can be hooked and held. Accordingly, the implant 20 and the elongated body 30 can be connected to each other through a simple operation in which the part of the elongated body 30 is hooked on the hook.

Also, the elongated body 30 is hooked in an annular shape on and attached to the hook. Accordingly, the implant 20 and the elongated body 30 can be separated from each other through a simple operation in which the part of the elongated body 30 is cut.

Also, the implant 20 is introduced into the living body 90 and the positioning operation is performed in astute before the expansive deformation. Accordingly, compared to a case where the positioning operation is performed after the expansive deformation, the implant 20 can be smoothly moved to the placement position.

### Modification Example of Puncture Member

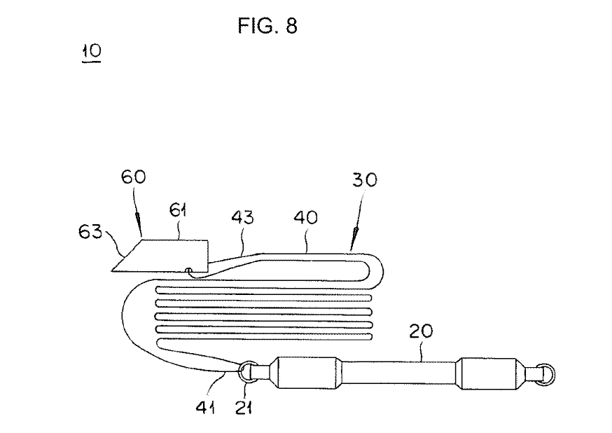

Next, referring to FIG. 8, a modification of the puncture member that assists in the introduction of the elongated body into the living body will be described.

In the above-described embodiment, an example in which the puncture needle that has the needle section 63 which is punctured into the living body 90, and a body section 65 into which the implant 20 can be inserted, is applied as the guide means 60 has been shown. However, for example, a puncture member 61 that does not have the body section 65 but has only the needle section 63 which can be punctured into the living body 90 can also be used as the guide means 60.

For example, as shown in FIG. 8, the implant assembly 10 is prepared in a state where a part of the elongated body 30 is connected to a proximal end of the puncture member 61. The connection is performed by passing the first elongated piece 40 as the elongated body 30 through the small hole disposed in the puncture member 61. In order to simplify a separation operation between the elongated body 30 and the implant 20, the elongated body 30 is hooked in an annular shape on the attachment section 21 of the implant 20.

Even in a case where the puncture member 61 according to the modification example is used, the elongated body 30 and the implant 20 can be introduced into the living body 90 with the puncturing of the puncture member 61 into the living body 90. Since the implant 20 and the elongated body 30 can be guided along the path in the living body 90 through which the puncture member 61 passes, the implant 20 can be moved to the vicinity of the placement position in a simple manner.

### Modification Example of Attachment Section

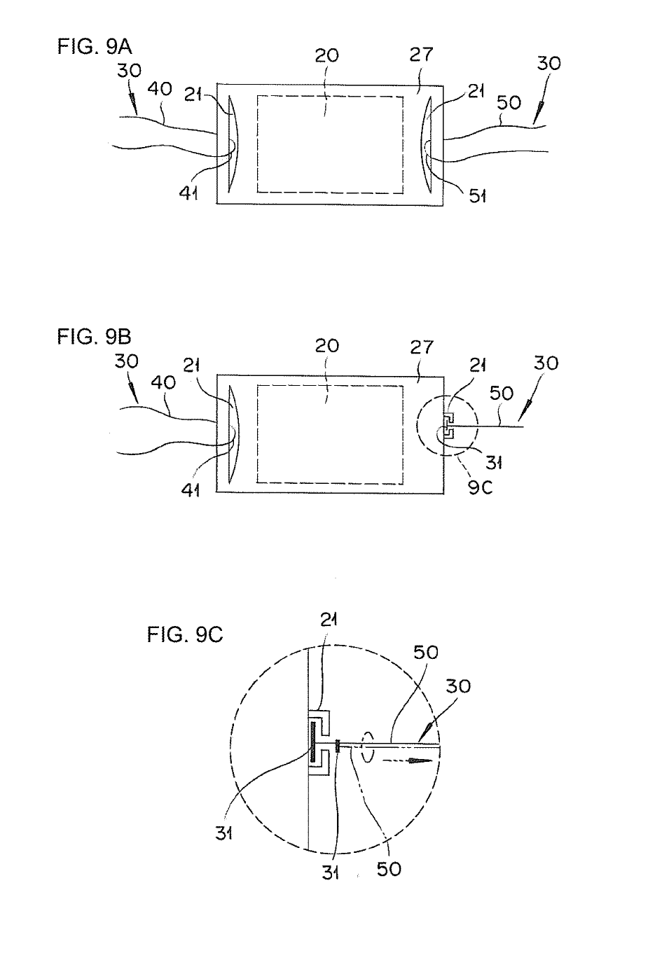

Next, referring to FIG. 9, a modification example of the attachment section of the implant to which the elongated body is attached will be described.

In the above-described embodiment, the ring-shaped attachment section 21 has been shown. However, the configuration of the attachment section 21 is not limited thereto, but can be appropriately changed as long as the elongated body 30 can be hooked and attached.

For example, as shown in FIG. 9A, a form in which a hole on which the elongated body 30 can be hooked to be connected is disposed in the implant 20 can be adopted. In the form that is shown, the hole forming the attachment section 21 is disposed in a cover member 27 where an IC chip and various medical instruments functioning as the implant 20 are accommodated so that a function of the attachment section 21 is added to the implant 20.

Also, for example, a hook from which the elongated body 30 can be detached can be adopted as the attachment section 21 as shown in FIG. 9B. In the form that is shown, a connection state is maintained by hooking a locking section 31 of the elongated body 30 through a hole disposed in a hook 21. The separation operation between the elongated body 30 and the implant 20 can be performed by rotating the elongated body 30 from the connection state and releasing locking with respect to the hook 21 (refer to FIG. 9C).

### Second Embodiment

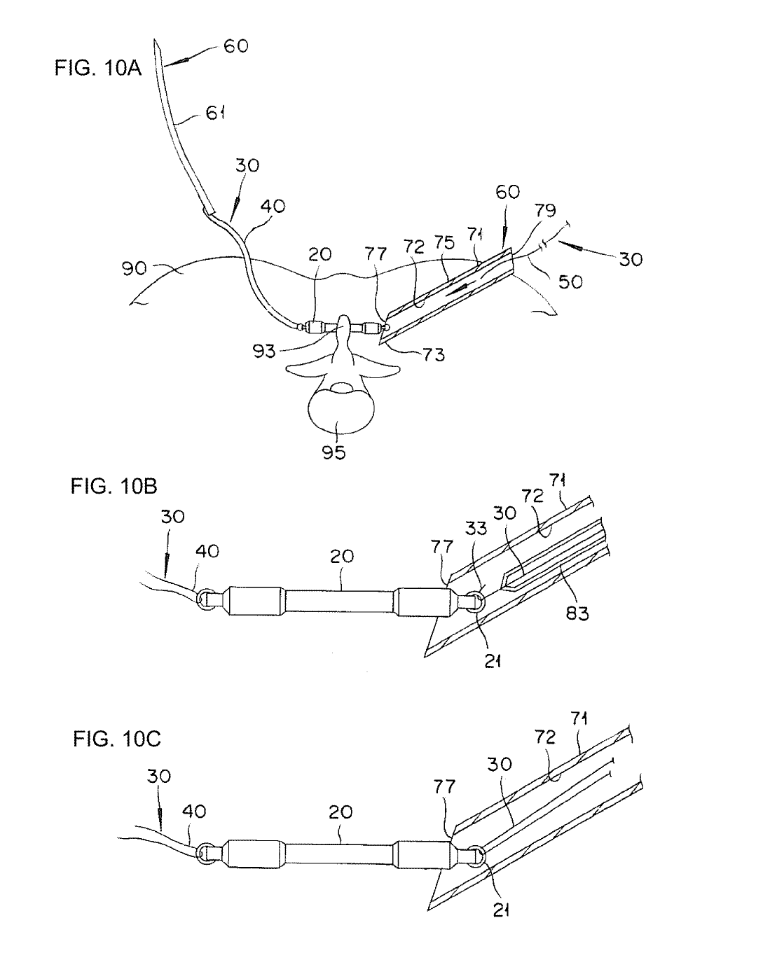

Next, referring to FIGS. 10A to 10C, a second embodiment of the present invention will be described. The same reference numerals are used to designate the same components as in the above-described embodiment and redundant descriptions thereof are omitted.

In the second embodiment, a tubular guide member 71 that has a lumen 72 into which the elongated body 30 can be inserted and opening sections 77 and 79 that are respectively formed on one end side and the other end side of the lumen 72 is used as the guide means 60 for assisting in the introduction of the elongated body 30 into the living body 90. The second embodiment is different in this point from the first embodiment in which the elongated body 30 is introduced by using the puncture member 61.

The tubular guide member 71 has a needle section 73 that is disposed at a tip, and a body section 75 where the lumen 72 is formed. The body section 75 is partially introduced into the living body 90 by puncturing the needle section 73 with respect to the living body 90. The opening section 77 that is positioned on a tip side of the tubular guide member 71 is provided in such a manner as to face the attachment section 21 of the implant 20, and the opening section 79 that is positioned on a proximal end side is provided outside the living body 90. In this state, the elongated body 30 is guided to the implant 20 via the lumen 72.

In order to introduce the tubular guide member 71 into the living body 90, a method in which a tip of a known medical instrument such as a guide wire is introduced into the living body 90 and then a proximal end of the guide wire is inserted into the lumen 72 of the tubular guide member 71 and the tubular guide member 71 is guided on the guide wire to the vicinity of the implant 20 can be adopted.

In an example that is shown in FIG. 10A, the implant 20 and the first elongated piece 40 are inserted into the living body 90 by using the puncture member 61 that has the body section 65, and then the second elongated piece 50 is introduced into the living body 90 by using the tubular guide member 71. Although the tube 25 is not shown in the drawing, the tube 25 is introduced into the living body 90 in a state, for example, of being connected to the implant 20 in advance as described in the first embodiment.

Referring to FIGS. 10B and 10C, a method in which the elongated body 30 is connected to the implant 20 in the living body 90 will be described.

For example, as shown in FIG. 10B, attachment can be performed by forming a bent section 33 at one end of the elongated body 30 and hooking the bent section 33 on the attachment section 21 of the implant 20. As shown in the drawing, the bent section 33 of the elongated body 30 can be connected to the implant 20 in a simple manner by using a gripper (for example, a pair of tweezers) 83 that can grip the elongated body 30 with the tubular guide member 71.

In the form of connecting the bent elongated body 30 to the implant 20, it is preferable that an elongated body formed from a metallic material, such as an SUS wire, a copper wire, a titanium wire, and a Nitinol wire, be used so that the bent shape of the tip is maintained.

After the positioning operation for the implant 20 by the elongated body 30 is completed, the tubular guide member 71 is withdrawn from the living body 90. The separation operation of the elongated body 30 from the implant 20 can be performed by introducing the tubular guide member 71 again into the living body 90 and via the lumen 72 of the tubular guide member 71 and the opening section 77 as is the case with the connection operation.

As shown in FIG. 10C, the connection with respect to the implant 20 can also be performed by hooking a part of the elongated body 30 without forming the bent section 33 in the elongated body 30.

In this manner, in a method for placing the implant according to the second embodiment, the step (i) includes a step of introducing the implant into the living body in a state of being separated from the elongated body, and the step (ii) includes a step of connecting the implant and the elongated body with each other in the living body after introducing the elongated body into the living body via the lumen of the tubular guide member. The step (vi) of separating the elongated body from the implant includes a step of separating the elongated body via the lumen and the opening section of the tubular guide member in a state where the opening section of the tubular guide member is provided in such a manner as to face the implant.

As in the embodiment, the connection and separation operations between the implant 20 and the elongated body 30 can be performed even after the implant 20 is introduced into the living body 90 by using the tubular guide member 71 as the guide means 60 for assisting in the introduction of the elongated body 30 into the living body 90. Since selection of an operation using the elongated body 30 can be appropriately determined in the medical field, convenience of the implant assembly 10 can be improved.

In the second embodiment, a form in which the puncture member 61 and the tubular guide member 71 are used in conjunction with each other is shown. However, the connection operation can be performed in such a manner that only the tubular guide member 71 is used to guide the first elongated piece 40 and the second elongated piece 50 to the implant 20 in the living body 90. In this case, the implant 20 can be introduced into the living body 90 ahead of the elongated body 30 by using a known medical instrument such as a guiding catheter.

The present invention can be appropriately modified.

In each of the embodiments, the two members having an elongated shape, that is, the first elongated piece 40 and the second elongated piece 50 are used as the elongated body 30. However, at least one elongated body may be provided so that the implant can be moved in the living body. Still, when a plurality of elongated pieces arc used as described in the embodiments, the implant can be moved in a plurality of directions and positioning accuracy can be improved. Also, for example, a form in which an additional elongated piece other than the two elongated pieces, that is, the first and second elongated pieces arc connected so that the implant can be moved in a further plurality of directions such as three directions and four directions can be appropriately adopted.

Also, a site to which the implant assembly is applied is not limited only to between the spinous processes in the living body, but the implant assembly can be applied to another site other than between the spinous processes. The implant is not particularly limited to those that are expansively and contractively deformed. In other words, an implant can be appropriately selected according to treatment details and application sites and an implant assembly forming a set with the elongated body and the guide means can be widely applied to each site of the living body.

Also, the method for attaching the implant and the elongated body with each other in a connectible and separable manner is not particularly limited to the form of hooking and holding the elongated body on and in the attachment section of the implant, but can be appropriately changed. For example, a connection form in which a screw section is disposed in the implant and one end of the elongated body is screwed can be adopted.

What is claimed is:

1. An implant assembly comprising:
an implant configured to be placed in a living body;
an elongated body that includes a pulling unit, the pulling unit being configured such that a portion of the pulling unit is locatable outside the living body when the implant is placed in the living body, and the pulling unit being connectable to the implant and pullable to move the implant to a placement position; and
a guide device configured to allow for a percutaneous introduction of the elongated body into the living body.

2. The implant assembly according to claim 1, wherein:
the elongated body includes at least a first elongated piece that is connected to a first portion of the implant, and a second elongated piece that is connected to a second portion of the implant, and
a pulling unit of the first elongated piece and a pulling unit of the second elongated piece are configured such that a portion of the first pulling unit and a portion of the second pulling unit are locatable outside the living body at different positions.

3. The implant assembly according to claim 1, wherein:
the guide device includes a puncture member configured to be percutaneously punctured into the living body,
the implant is connected to the puncture member via the elongated body and is configured to be introduced into the living body as the puncture member is punctured into the living body, and
the elongated body is configured to be introduced into the living body with the implant while the elongated body is connected to the implant.

4. The implant assembly according to claim 3, wherein:
the puncture member has a body section into which the implant is insertable, and
the implant is configured to be introduced into the living body while the implant is located in the body section.

5. The implant assembly according to claim 1, wherein the guide device includes a tubular guide member which has a lumen into which the elongated body is insertable, a first opening located on a first side of the lumen, and a second opening located on a second side of the lumen.

6. The implant assembly according to claim 1, wherein the implant includes an attachment section to which the elongated body is attached in a connectible and separable manner.

7. The implant assembly according to claim 6, wherein:
the attachment section includes at least one hook by which the elongated body is hooked and held, and
the implant and the elongated body are connectable to each other via the at least one hook.

8. The implant assembly according to claim 7, wherein the elongated body has an annular shape and is separable from the implant by cutting a portion of the elongated body.

9. The implant assembly according to claim 1, wherein the implant is an expansion member that is expansively deformable by an introduction of a filling material and is introducible into the living body in a state before the expansive deformation.
